(19) United States
(12) Patent Application Publication
(10) Pub. No.: US 2004/0254445 A1
(43) Pub. Date: Dec. 16, 2004

(54) Use of MRI to screen individuals for prostate cancer

(76) Inventor: Craig Bittner, Scottsdale, AZ (US)

Correspondence Address: PATTON BOGGS LLP, 8484 WESTPARK DRIVE, SUITE 900, MCLEAN, VA, 22102, US

(21) Appl. No.: 10/460,798

(22) Filed: Jun. 13, 2003

(51) Int. Cl. A61B 5/05

(52) U.S. Cl. 600/410

(57) ABSTRACT

Methods of screening individuals for prostate cancer are provided, which may enable early diagnosis of prostate cancer. The methods may involve the use of a contrast agent while screening individuals using MRI technology. The methods may involve the use of MRI spectroscopy while screening individuals using MRI technology. Also provided are methods of detecting prostate cancer in individuals, which include screening such individuals using MRI technology.

### FIELD OF THE INVENTION

The present invention relates to methods of screening individuals for prostate cancer, and to methods of detecting prostate cancer in individuals using Magnetic Resonance Imaging (MRI) technology.

### BACKGROUND OF THE INVENTION

Prostate cancer is the second leading cause of male cancer death in the United States exceeded only by lung cancer. An estimated 28,900 men will die from prostate cancer this year in the U.S. The American Cancer Society estimates that there will be about 220,900 new cases of prostate cancer in 2003. Prostate cancer is the most commonly diagnosed cancer in America among men after skin cancer. One in every six men will get prostate cancer during his life. The chances of getting prostate cancer are one in three if an individual has one close relative (father, brother) with the disease. The risk increases five-fold with two close relatives. With three, it is an almost certainty (97%) that an individual will get prostate cancer. (National Prostate Cancer Coalition). Only 1 man in 32 will die of prostate cancer, and the death rate decreases as the disease is being found earlier. Nearly 100% of men with prostate cancer that is detected early, can survive.

The prostate gland is part of the male reproductive system. Cancerous prostate tumors can, if untreated, spread to other parts of the body. Often there are no symptoms, which is why screening is so important. With early detection, prostate cancer is one of the most curable forms of cancer.

The American Urological Association (AUA) encourages physicians to routinely offer prostate cancer testing to men who have an anticipated lifespan of 10 or more years and are: over the age of 50 years; or over the age of 40 years old have a family history of the disease (for example, a father or brother who was diagnosed with prostate cancer), or over the age of 40 years and African-American.

There are two widely used tests to aid in the early detection of prostate cancer. The PSA test is a blood test, which measures the level of protein called prostate-specific antigen (PSA). Normally, PSA is found in the blood at very low levels. Elevated PSA readings can be a sign of prostate cancer; however, PSA levels can be elevated for reasons other than cancer such as due to Benign Prostatic Hypertrophy (BPH). Only about 20% of men with a PSA level greater than or equal to 4 ng per mL will have prostate cancer (Asian J. Surg. 2002 July; 25(3):238-43 and Ann Intern Med 1997 March 1; 126(5): 394-406). The majority of men with elevated PSA levels will have only BPH or other benign changes of the prostate. This indicates a high lack of specificity with the PSA test.

In addition to having low specificity, the PSA test often misses prostate cancer. In a study of over 11,000 men with PSA testing, prostate cancer was missed in up to 25% of men. (Eur Urd 2001; February; 39(2):131-7). Other studies demonstrated that PSA will miss 1 out of 3 men with prostate cancer, because over one third of all prostate cancers would not have an elevated PSA (i.e., would be below 4 ng/ml). (J. Urol. 2000 March; 163(3): 806-12 and J. Clin. Lab. Anal. 1993; 7(5):283-92). Even when PSA may indicate that prostate cancer might be present, the cancer might be quite advanced. Men who have a mild elevation of PSA may already have advanced prostate cancer. In men with a PSA of 4 to 10 ng/ml, 40% may already have extra capsular spread beyond the prostate (CA Cancer J. Clin 1992 July-August; 42(4):198-211).

The second test is a physical exam or digital rectal exam (DRE). DRE is a slightly uncomfortable physical exam performed by a physician. DRE is performed in attempt to palpate any abnormalities in the contour or consistency of the posterior aspect of the prostate. Like the PSA, the DRE can fail to detect up to 80% of all prostate cancers (Kaohsiung J Med Sci. 2002 June; 18(6):281-8). The DRE is not able to detect prostate cancers that are not palpable and often the DRE is abnormal due to benign changes in the prostate from BPH.

There can be cancers that will be missed by PSA, which might be detected by the DRE. Currently, the most comprehensive method for early detection uses both the PSA and DRE tests. These methods do not however, provide an adequate, sufficiently accurate, early detection of prostate cancer.

### SUMMARY OF THE INVENTION

The present invention provides methods of screening individuals for prostate cancer using MRI technology and methods of detecting prostate cancer in individuals, which include screening such individuals using MRI technology. According to embodiments of the invention, the methods may involve the use of a contrast agent while screening individuals using MRI technology. According to embodiments of the invention, these methods may involve the use of MRI spectroscopy while screening individuals using MRI technology.

The present invention also provides methods of detecting prostate cancer in individuals, which include screening such individuals using MRI spectroscopy technology.

The present invention may enable more accurate and earlier diagnosis of prostate cancer.

### BRIEF DESCRIPTION OF THE DRAWINGS

FIG. 1 is a flow chart representative of an algorithm showing assessment of diagnostic information obtained using MRI to screen individuals for prostate cancer.

FIG. 2 is a sample curve demonstrating relative enhancement over time for a region of cancer within a prostate.

FIGS. 3-4 are screen captures of examples of how the control panel of the MRI may be set up for imaging protocols in accordance with the methods of the present invention.

FIGS. 5-16 depict sample MRI images of an individual according to the methods of the present invention. The individual did not show any region suspicious for cancer.

FIGS. 17-30 depict sample MRI images of an individual according to methods of the present invention. This individual had prostate cancer.

### DETAILED DESCRIPTION OF THE INVENTION

This application relates to the following patent application filed concurrently herewith, which is incorporated by reference herein in its entirety: “Use of MRI to Screen Normal Risk, Asymptomatic Individuals for Breast Cancer,” (attorney docket # 011339.0105).

There is no sufficiently effective method for the screening of individuals for the presence of prostate cancer. Methods of screening such as the PSA blood test or the digital rectal examination are inaccurate. This is especially the case in individuals having no symptoms. None of the known detection methods is a successful method for screening individuals for prostate cancer. The PSA test and DRE will miss approximately 30 to 50% of men with prostate cancer. There is a need for an effective and more accurate method of screening individuals for the presence of prostate cancer. Availability of an easy to employ, more accurate methodology for regular testing will lead to vast improvements in early and accurate diagnosing and lowering of the morbidity and mortality of prostate cancer.

Accordingly, the present invention provides methods for screening individuals for prostate cancer using MRI technology. In particular, the methods include performing MRI on individuals; and determining from the MRI whether those individuals have indications of prostate cancer.

The present invention also provides methods of detecting prostate cancer in individuals, which include screening individuals for prostate cancer, where the screening includes performing MRI on the individuals; and determining from the MRI whether those individuals have indications of prostate cancer.

According to embodiments of the invention, the individuals may be at normal risk for developing prostate cancer and/or may be asymptomatic for prostate cancer. As well, the individuals may have an abnormality on one of the other screening tests, such as an abnormal DRE or elevated PSA level, but have not yet had a prostate biopsy rendering a diagnosis of prostate cancer.

Normal risk individuals are those who do not have a significant family history of prostate cancer and have not otherwise been told by a physician that for whatever reason they have a high risk of acquiring prostate cancer.

Asymptomatic individuals are those who do have any of the symptoms of prostate cancer including for example difficulty starting urination, reduced force of stream of urine, urinating small amounts frequently, urinating more frequently during the night, painful urination and bone pain.

Because PSA level is not determinative as to the presence, severity or location of any prostate cancer, and because the PSA test lacks specificity, it is not an accurate screening method. According to embodiments of the invention, the present invention provides methods of screening individuals having an elevated PSA level for prostate cancer where the screening includes performing MRI on the individuals and determining from the MRI whether those individuals have indications of prostate cancer.

Because an abnormal DRE is not determinative as to the presence, severity or location of any prostate cancer, and because the DRE test lacks specificity, it is not an accurate screening method. According to embodiments of the invention, the present invention provides methods of screening individuals having an abnormal DRE for prostate cancer where the screening includes performing MRI on the individuals and determining from the MRI whether those individuals have indications of prostate cancer.

Embodiments of the invention also relate to methods of determining one or more locations of prostate cancer within the prostate by performing MRI on the prostate.

Embodiments of the invention also relate to methods of guiding a biopsy to a cancer within the prostate by performing MRI on the prostate and guiding the biopsy to a desired location using images produced by the MRI and known biopsy techniques. MRI may be a significantly more accurate guidance method than ultrasound guidance, in which prostate cancer may be easily missed. For example, in a study of over 2,500 men, nearly one in four prostate cancers were missed using ultrasound guided prostate biopsy (J. Urol. 2002, June; 167(6):2435-9).

Magnetic resonance imaging (MRI) has become an important non-invasive medical technique over the past decade. U.S. Pat. No. 6,363,275, which is hereby incorporated by reference, discloses a device for detecting and for treating tumors using differential diagnosis.

MRI uses a strong direct current magnetic field in conjunction with tunable gradient magnetic fields to spatially control locations at which the net sum magnetic field reaches a preselected value. As the magnetic bias fields are varied spatially, a series of radio frequency (RF) pulses are applied. When the RF energy is at a resonance frequency of sample atoms, of a particular species and surroundings, those sample atomic nuclei absorb the RF energy and are excited to a higher spin state. The excited spin state then decays to a lower energy state of excitation and the decay is accompanied by an emission of an RF pulse, known as “spin echo.” The RF of a nucleus and its resulting spin echo signal depend on a number of factors, including mass, density, dipole moment, relaxation frequency, as well as chemical bonding and electrostatic potential of its surroundings.

According to embodiments of the invention, the methods of the invention utilize MRI technology. MRI is performed of the prostate. Images of the prostate are acquired using various MRI sequences, such as T1, T2 and Proton Weighted images of the prostate. The images demonstrate the internal structure of the prostate tissue and various specific MRI protocols can be used to accentuate the image characteristics of various regions within the prostate such as the central zone, the transitional zone and the peripheral zone as well as various abnormalities that may be present such as prostate cancer. The images can be reviewed by a board-certified radiologist or other individual utilizing specialized medical workstations that help evaluate the image data to aid in the detection of prostate cancer even at its earliest stages. The radiologist or other interpreting individual and workstations may be either on-site at the location where the MRIs are being performed, or they may be at a remote location to where the MRI images are sent.

According to embodiments of the invention, the disclosed methods may involve the use of a contrast agent while screening individuals using MRI technology. In particular, in order to enhance the contrast between tissues within an organism, one or more contrast agents may be introduced into an individual's body prior to MRI analysis. Contrast agents, which may be used in accordance with the present invention include suitable contrast agents known to those in the art, including for example, paramagnetic metal ions such as manganese, gadolinium and iron.

The large dipoles associated with paramagnetic ions, as compared to protons, perturbs the proton's relaxation time, T, as a function of distance between the ion and the proton, thus providing a strong differential signal which can be used for imaging. WO 02/13874, which is hereby incorporated by reference, discloses the use of metal complexes containing perfluoroalkyl as contrast agents in MRI for the representation of tumoral tissue. WO 01/82976, WO 00/16811 and WO 99/26535, which are also hereby incorporated by reference, disclose MRI contrast imaging agents for imaging cancer. These contrast agents may be used in accordance with the present invention.

The methods of the invention are novel in the way they examine the function of the prostate tissue as well as its structure. The PSA blood test does not currently have this capability. For a cancer to grow it must establish its own blood supply in a process called angiogenesis. In angiogenesis, the cancer will form new blood vessels that are unlike any normally found in the prostate of adults. In particular, these angiogenic blood vessels demonstrate abnormal physiologic function, which is distinguishable from that of normal adult blood vessels found in non-cancerous tissue. Thus, according to embodiments of the invention, utilizing a special technique called Dynamic Enhancement Imaging (DEI), MRI can evaluate the prostate tissue for the abnormal function of these angiogenic blood vessels.

In accordance with certain embodiments of the methods of the invention, possible sites of prostate cancer in an individual's prostate can be identified with the aid of MRI by means of a dynamic investigation in which the contrast enhancement behavior of the prostate is evaluated. The contrast agent may be introduced by injection into the arm of an individual (or introduced by other suitable means such as injection into a vein or artery elsewhere in the body) in a relatively small dose on the order of approximately 10-30 mL, approximately 15-25 mL, or approximately 20 mL. A secondary injection (or other form of administration) into the individual of a physiological saline solution or other suitable fluid of about the same volume as that of the contrast agent may also be used so that the contrast agent reaches the blood circulation as completely as possible.

According to embodiments of the invention, a baseline pre-contrast MRI of the prostate is performed, and then a contrast agent is administered in a dosage of approximately 0.07-0.13 mmol/kg, 0.085-0.115 mmol/kg, or approximately 0.1 mmol/kg of body weight of the individual. By performing a dynamically enhanced MRI with T1 weighting of the prostate over a period of approximately 2 to 10 minutes, approximately 3 to 8 minutes, or approximately 5 minutes at specific intervals, such as intervals of approximately every 5 to 90 seconds, or approximately every 60 seconds, cancer from normal prostate tissue can be differentiated with a very high degree of sensitivity and specificity. The dynamically enhanced MRI may be started within approximately 0 to 120 seconds or approximately 40 seconds from the time the contrast agent is being injected. The contrast agent is administered at a bolus rate of approximately 1 to 3 cc/sec or approximately 2 cc/sec and subsequent MRI imaging through the prostate is then acquired at approximately 5 second to 90 second intervals, or approximately 60 second intervals. By this method, the degree of percentage of MRI signal enhancement over time may be evaluated and a percentage of enhancement versus time curve plotted. Regions of cancer growth within the prostate demonstrate a significantly higher degree of enhancement than surrounding normal prostate tissue early after contrast agent administration and this degree of enhancement tends to decrease much more quickly than that of the normal tissue. This difference in enhancement is a direct effect of the differing physiology of the cancer's angiogenic blood vessels.

Accordingly, the present invention also includes a method of screening an individual for prostate cancer, which includes obtaining a baseline pre-contrast MRI of an entire prostate of an individual, administering a contrast agent to the individual in an amount of approximately 0.07-0.13 mmol/kg, 0.085-0.115 mmol/kg, or approximately 0.1 mmol/kg body weight of the individual, performing a dynamically enhanced MRI with T1 weighting of the prostate over a period of approximately 2 to 10 minutes, approximately 3 to 8 minutes, or approximately 5 minutes at intervals of approximately every 5 to 90 seconds, evaluating and possibly plotting a degree of percentage of MRI signal enhancement over time, and identifying any regions of possible cancer from that of normal prostate tissue based on the degree of enhancement in addition to the structural characteristics of the tissues present in the MRI images.

T1 weighting is a description of any MRI protocol that accentuates the differing longitudinal magnetization relaxation of protons. MRI protocols can also be employed that accentuate the T2 weighting of various protons by accentuating their differing transverse magnetization characteristics or that accentuate the proton density of tissue by minimizing both the T1 and T2 weighting within the MRI protocols. These differences are achieved by altering the TR (Time of Repetition) and TE (Time of Echo) of the RF (radio frequency) pulses used to excite the protons to an excited spin state, and their behavior is determined by their inherent T1 and T2 relaxation (or decay) properties.

T2 weighted images and Proton Density weighted images may also be gathered in accordance with the present invention, which may be useful in identifying a structural abnormality within the prostate tissue and in distinguishing cancer from benign tissue such as for example distinguishing a cancer from benign prostatic hyperplasia. This information may be used in conjunction with the information obtained in the dynamic enhancement images such as plotting relative MRI signal enhancement over time for an area of structural abnormality seen in the non-dynamic enhanced images.

As with other methods of the invention, a board certified radiologist or other individual identifies any regions of possible cancer growth from normal prostate tissue possibly utilizing specialized medical workstations that help to evaluate the information obtained in the dynamic contrast images and other MRI images. The radiologists and workstations may be either on-site at the location where the MRIs are being performed, or they may be at a remote location to where the MRI images are sent.

The contrast agent may include one or more suitable contrast agents, such as those described above, which include, but are not limited to paramagnetic metal ions, including for example, manganese, gadolinium and/or iron. According to these methods, the contrast agent may be administered at a bolus rate of approximately 1 to 3 cc/sec or approximately 2 cc/sec.

According to embodiments of the invention, the disclosed methods may involve the use of MRI spectroscopy while screening individuals using MRI technology.

MRI spectroscopy can non-invasively obtain physiologic images and spectra, based on the relative concentrations of cellular chemicals and metabolites. MRI spectroscopy can provide physiologic information about the relative concentrations of metabolites such as, citrate, creatine, and choline within the prostatic tissue by measuring the specific resonances for the metabolites from small volumes of tissue throughout the prostate. The amount of individual resonance present for each metabolite is related to the concentration of these metabolites and changes in these concentrations can be used to identify prostate cancer from normal prostate tissue and from non-cancerous changes of the prostate tissue. For example, prostate cancer will demonstrate significantly higher choline levels and significantly lower citrate levels as compared to normal prostatic tissues and benign changes in the prostate. The ratio of these metabolites (e.g., choline/citrate) in regions of cancer have minimal overlap with values of normal prostate tissue and BPH values. Also, the physiologic information obtained from MRI spectroscopy may enable assessment of cancer aggressiveness.

Accordingly, the present invention provides methods of detecting prostate cancer in individuals, which include screening individuals for prostate cancer, where the screening includes performing MRI spectroscopy on one or more individuals; and determining from the MRI spectroscopy whether those individuals have indications of prostate cancer.

The present invention further provides methods of screening individuals for prostate cancer, which include obtaining a MRI spectroscopy of a prostate of an individual, determining relative concentrations of one or more metabolites within the prostatic tissue of the prostate by measuring specific resonances of the metabolites from small volumes of tissue throughout the prostate, determining ratios of the metabolites in the prostate, and differentiating regions of cancer growth from normal prostate tissue based, inter alia, on the ratios. The metaboites may include one or more of the following exemplary metabolites, citrate, creatine and choline, and other metabolites known to those skilled in the art. Small volumes of tissue may include volumes of, for example, approximately 1 mm³ to 1 cm³ from various portions throughout the prostate as determined by a skilled practitioner.

The present methods of detecting prostate cancer and screening individuals using MRI have significant advantages in the field of prostate cancer screening, including e.g.: (1) the improved ability to detect early prostate cancers; (2) the improved ability to differentiae between prostate cancer and normal prostate tissue; (3) the improved ability to differentiate between prostate cancer and benign lesions in the prostate; (4) the improved ability to differentiate between scar and recurrent prostate cancer; (5) the improved detection of multifocal and/or multicentric prostate cancers; (6) the improved ability to determine the extent of prostate cancer present; and (7) the utilization of a less invasive procedure than DRE.

The methods of the invention are significantly better at detecting early prostate cancer than PSA or DRE, and thus, will detect early cancers that PSA or DRE may miss. Because the methods of the invention are more accurate, individuals who may have undergone unnecessary biopsies for benign changes of the prostate detected by PSA or DRE, may not have to undergo such procedures had they been screened using the present method, which are better able to distinguish cancerous from benign abnormalities. The methods of the invention also permit detection of lesions located deep within the prostate tissue, which are often missed during DRE.

The diagnostic benefits arising from the present invention include the overall management of prostate cancer in men. The diagnosis of prostate cancer at an earlier stage allows an individual more choices in selection of various treatment options and a much higher probability of survival.

The methods of the invention may also result in reduced healthcare costs by changing the focus of medicine from expensive and reactive cancer treatment to cost-effective, proactive early detection. The methods of the invention, with its dramatically improved accuracy, could prevent unnecessary biopsies, saving healthcare dollars, as well as detecting prostate cancer in individuals early enough that the cancer may be removed without the need for adjuvant therapy such as radiation, thus preventing this additional needless emotional and physical suffering of thousands of individuals.

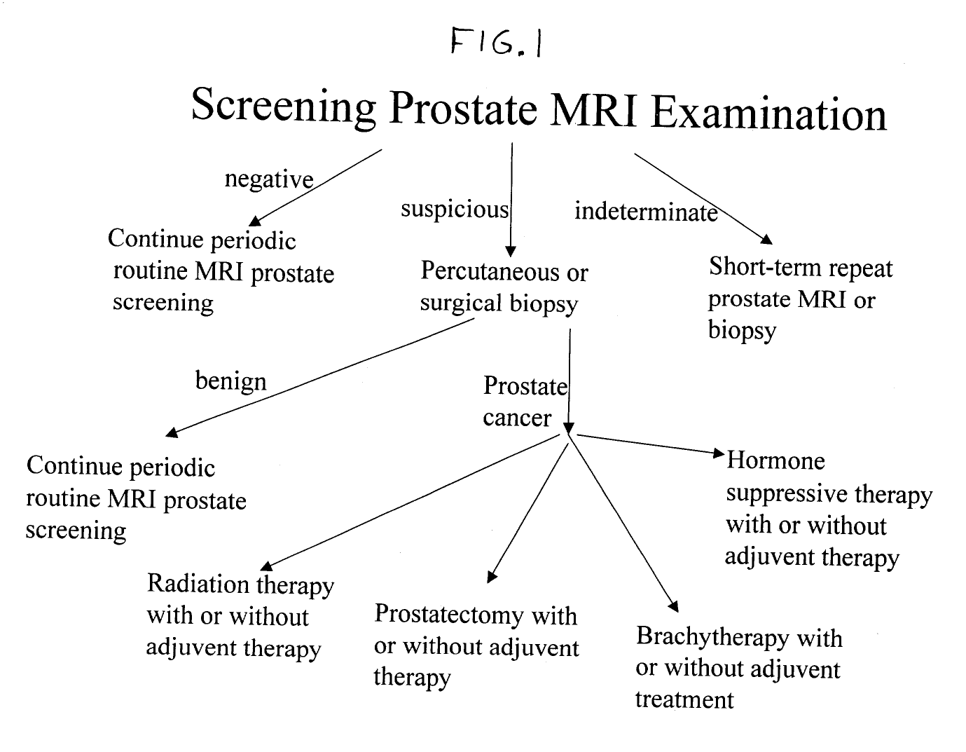

FIG. 1 is a flow chart representative of an algorithm showing assessment of diagnostic information obtained using MRI to screen individuals for prostate cancer. As depicted in FIG. 1, individuals are first screened for prostate cancer using MRI in accordance with the methods of the present invention. If the screening is negative, i.e., shows no indications of prostate cancer, the individual should continue periodic (e.g., yearly depending on age) routine MRI prostate screening. If the screening results in some indication of suspicious tissue or physiological activity, then a percutaneous or surgical biopsy should be performed. If the biopsy indicates that the suspicious tissue or lump(s) is benign, the individual should continue periodic (e.g., yearly depending on age) routine MRI prostate screening. If the biopsy indicates that the suspicious tissue or lump(s) is prostate cancer, then depending on the size of the lump, spread of the cancer, and other factors, the physician may recommend one or more of several treatment options including, for example, (1) prostatectomy surgery with or without adjuvent therapy; (2) cryosurgery with or without adjuvent therapy; or (3) brachytherapy (radioactive seed implantation) with or without adjuvant therapy; (4) induction radiation therapy followed by prostatectomy surgical therapy with or without adjuvent treatment; (5) induction hormonal suppression therapy followed by prostatectomy surgical therapy with or without adjuvent treatment; (6) induction radiation therapy followed by cryosurgical therapy with or without adjuvent treatment; (7) induction hormonal suppression therapy followed by cryosurgical therapy with or without adjuvent treatment; (8) induction radiation therapy followed by brachytherapy with or without adjuvent treatment; (9) induction hormonal suppression therapy followed by brachytherapy with or without adjuvent treatment; (10) external radiation therapy; and (11) hormonal suppression therapy. Adjuvent therapy or treatment means supplementary treatment such as performing radiation therapy or chemotherapy in addition to surgical intervention. If the screening is indeterminate, i.e., it is not suspicious, but it cannot be said to show no indications of prostate cancer, the individual should repeat the prostate MRI within a short period of time (e.g., within 3 to 6 months of the first MRI) or have a percutaneous or surgical biopsy performed as described above.

The following examples illustrate specific embodiments of the invention. The examples set forth herein are meant to be illustrative and should not in any way serve to limit the scope of the claimed invention. As would be apparent to skilled artisans, various changes and modifications are possible and are contemplated within the scope of the invention described.

### EXAMPLES

### Example 1

An examination is conducted during which MRI is performed on the prostate of an individual. Several images of the prostate are acquired using various MRI sequences to produce T1, T2 and/or Proton Weighted images of the prostate. The images are then reviewed by a board-certified radiologist utilizing specialized medical workstations, which detect any indications of prostate cancer.

### Example 2

A dynamic investigation is performed on an individual, in which the enhancement behavior of a contrast agent is evaluated. An individual may rest on their back on a MRI scanner table. A baseline pre-contrast MRI of the entire prostate is performed with a T1 weighted image sequence. A small IV may be placed in an arm vein of the individual and a gadolinium contrast agent is injected into the arm in a dose of approximately 20 mL. The individual is then injected with a physiological saline solution of about the same amount (i.e., approximately 20 mL), so that the contrast agent reaches the blood circulation as completely as possible.

By administering intravenous gadolinium in a dosage around 0.1 mmol/kg of body weight and performing a dynamically enhanced MRI with T1 weighting of the prostate over a period of minutes at specific intervals, cancer from normal prostate tissue can be differentiated with a very high degree of sensitivity and specificity.

The gadolinium contrast agent is administered at a bolus rate of approximately 2 cc/sec and subsequent MRI imaging through the prostate is then acquired at 1 minute intervals. In so doing the degree of percentage of MRI signal enhancement over time is plotted. Regions of cancer growth within the prostate demonstrate a significantly higher degree of enhancement than surrounding normal prostate tissue and this degree of enhancement decreases much more quickly than that of the normal tissue. A board certified radiologist identifies any regions of possible cancer from the normal prostate tissue based on the degree of enhancement in addition to the structural characteristics of the tissues present in the MRI images.

### Example 3

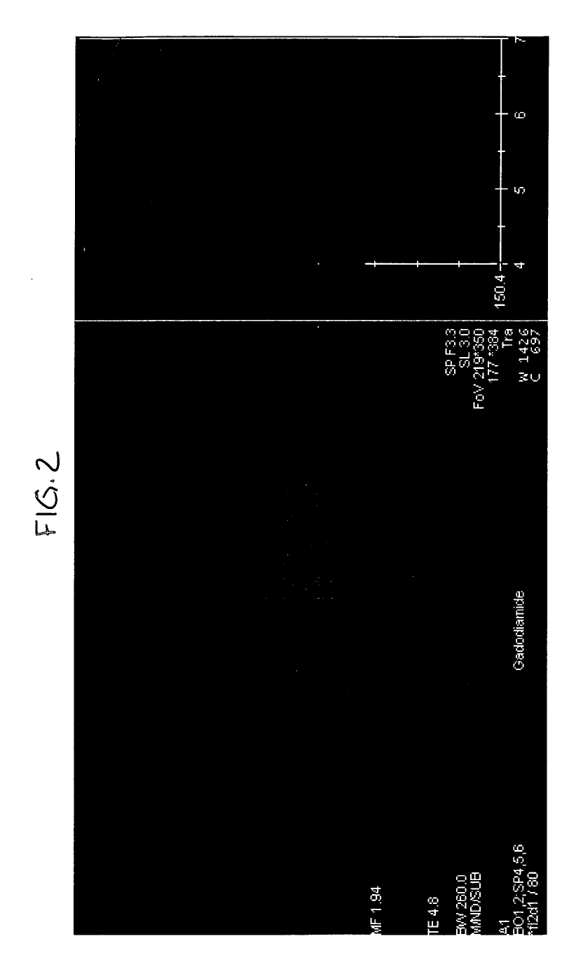

The prostate of an individual was examined using MRI in accordance with the methods of the present invention. FIG. 2 is a sample curve demonstrating relative enhancement over time for a region of cancer within a prostate. FIG. 2 demonstrates the relative high degree of contrast enhancement early after injection with a contrast agent, which contrast enhancement decreases shortly thereafter.

### Example 4

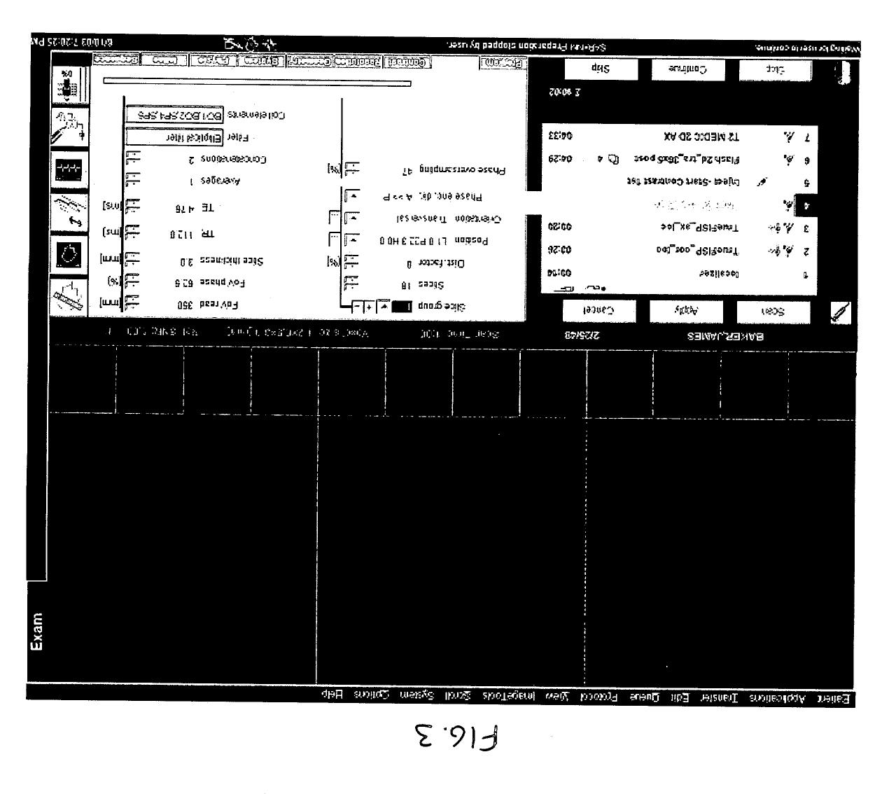

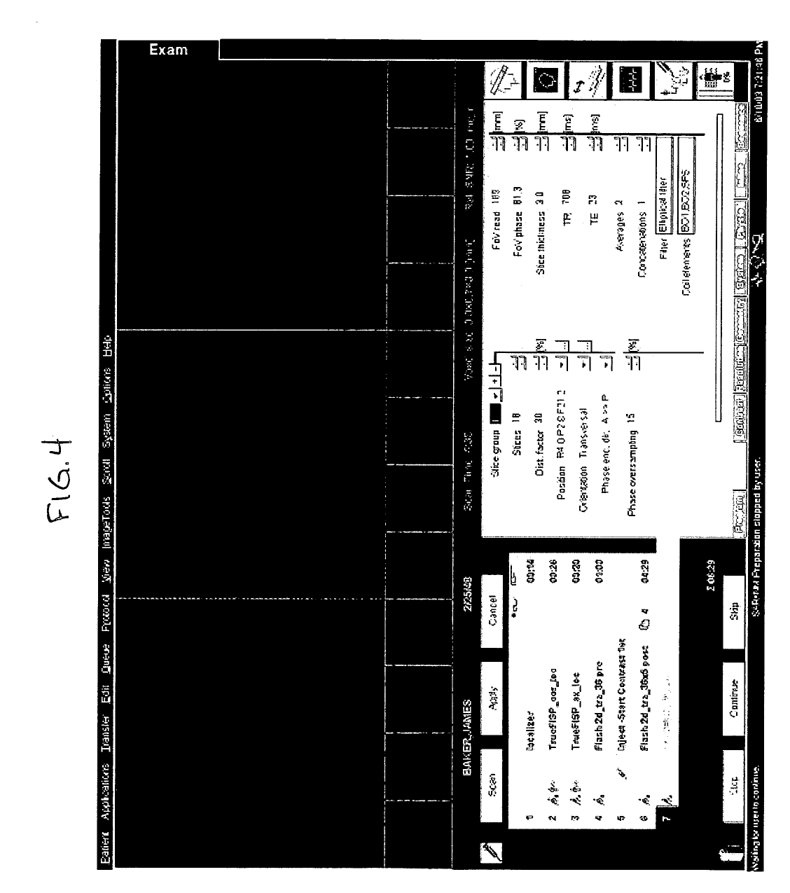

The control panel of an MRI may be set up as depicted in FIGS. 3-4, for imaging protocols in accordance with the methods of the invention. FIG. 3 is an image depicting the ‘task card’ in the bottom right corner that demonstrates all of the various setting such as the TE, TR, etc . . . for the pre-contrast images. The MRI may be set up such that the task card is the same for post contrast images. FIG. 4 depicts the ‘task card’ settings for a T2 sequence in accordance with methods of the present invention.

### Example 5

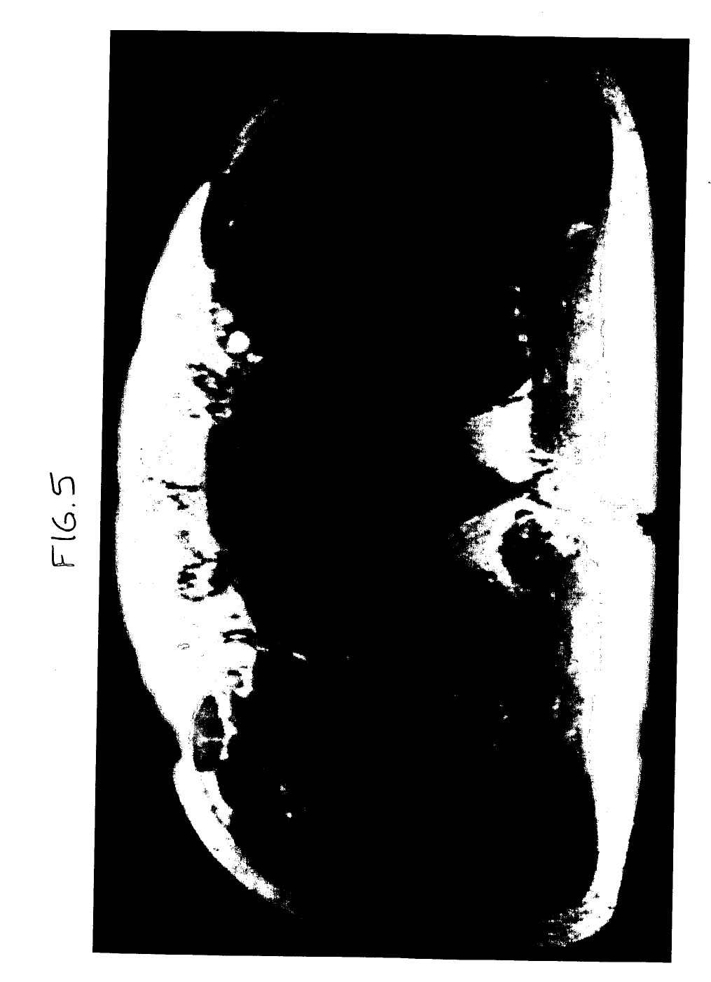

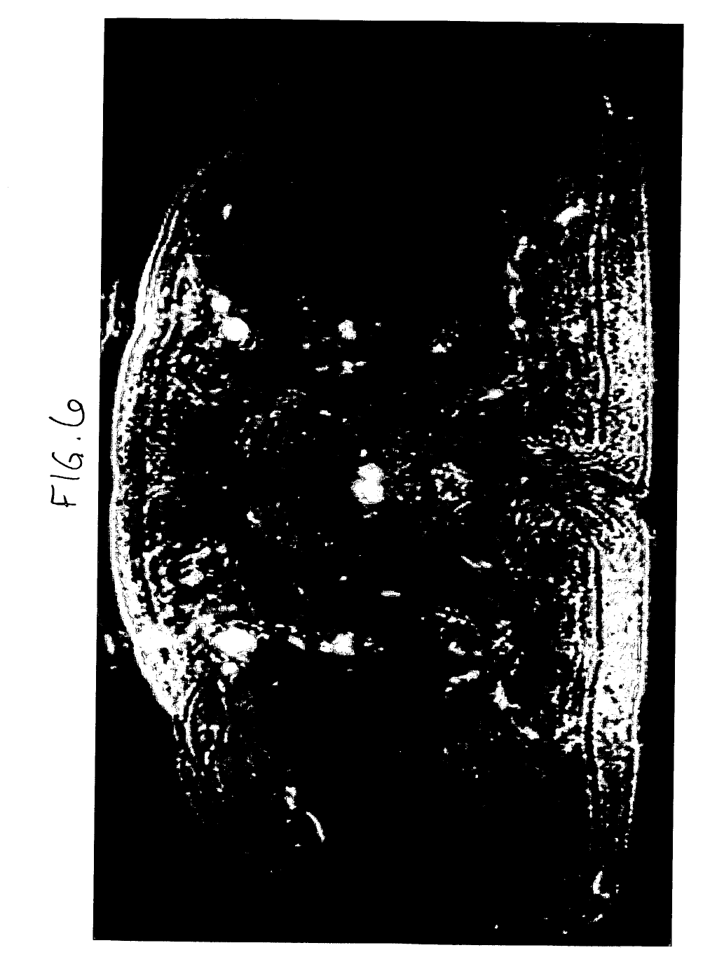

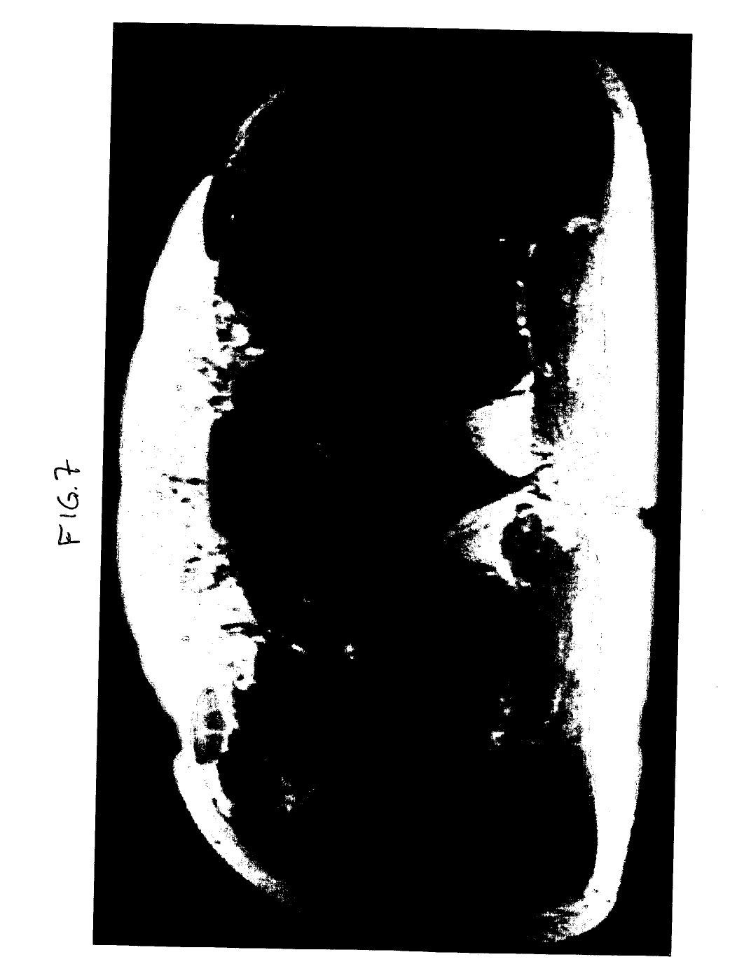

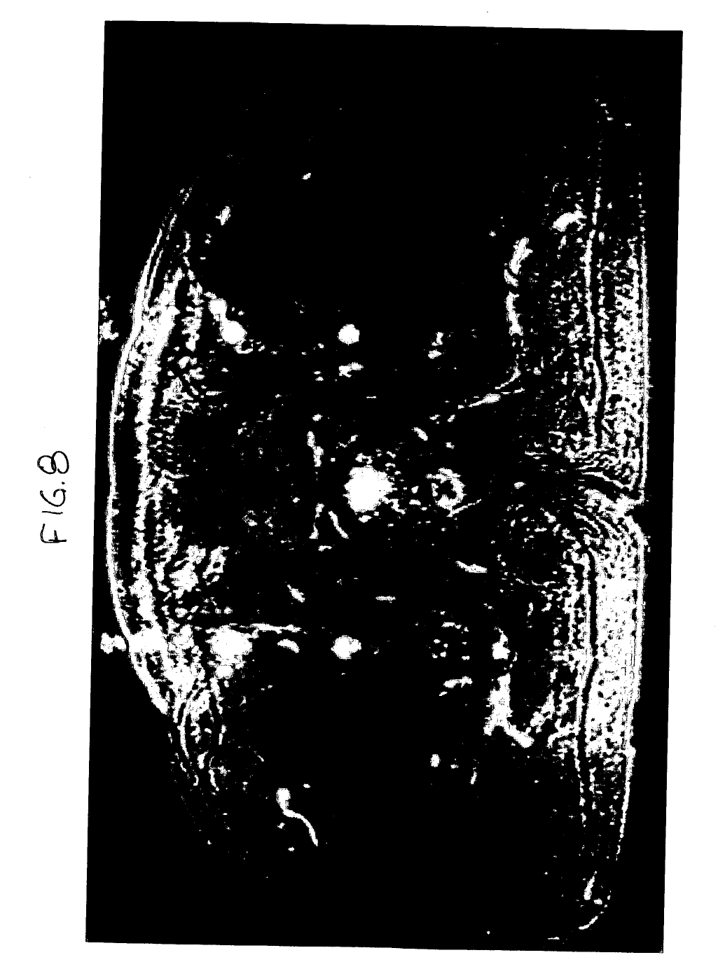

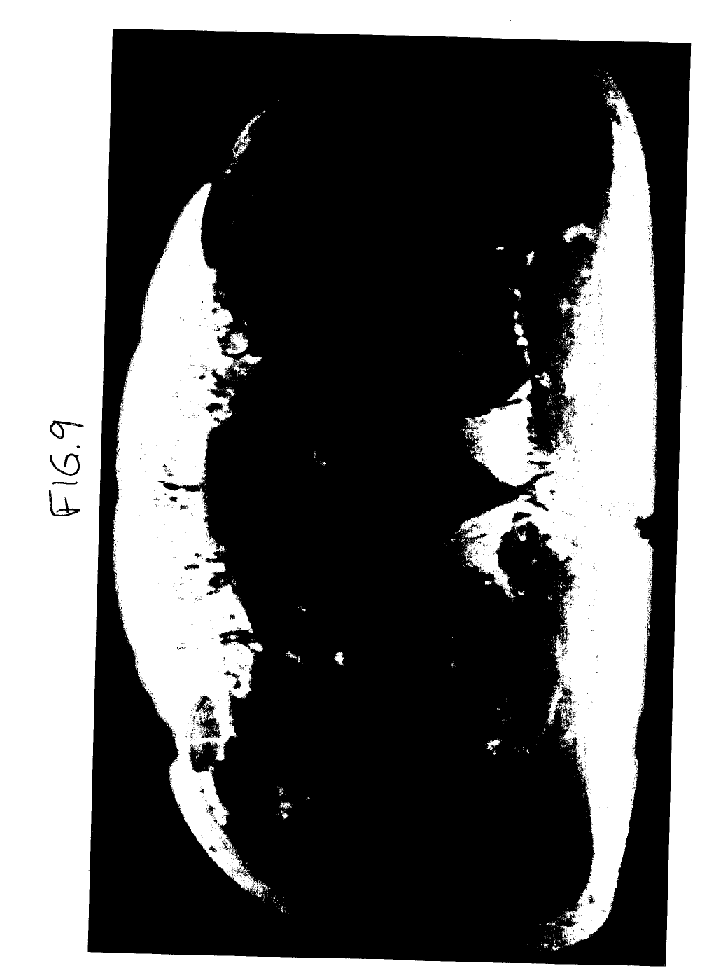

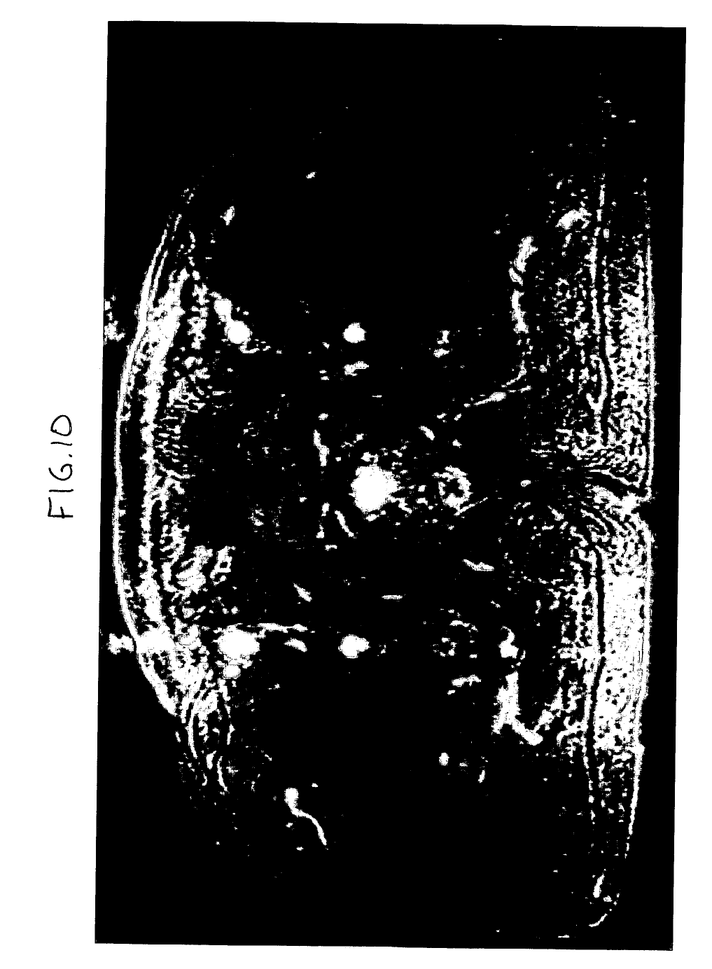

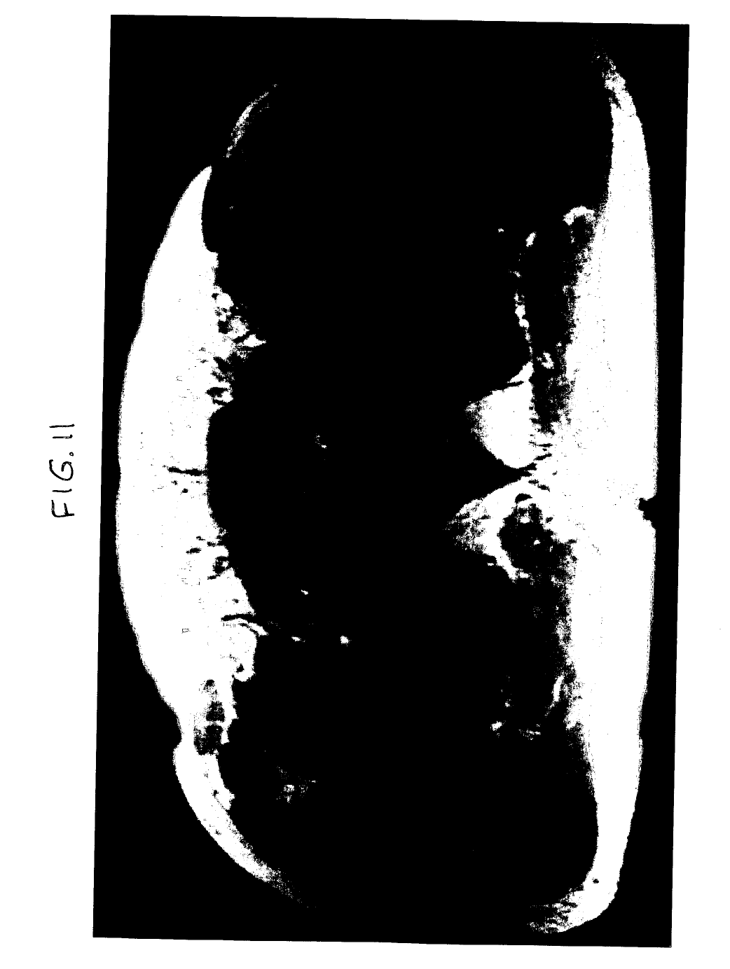

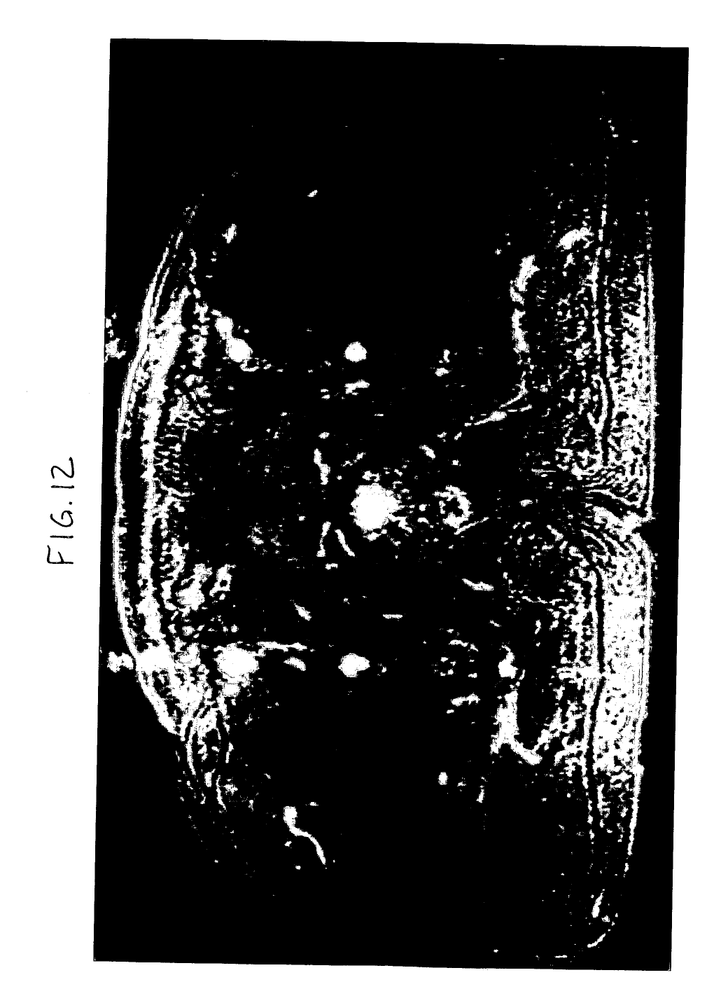

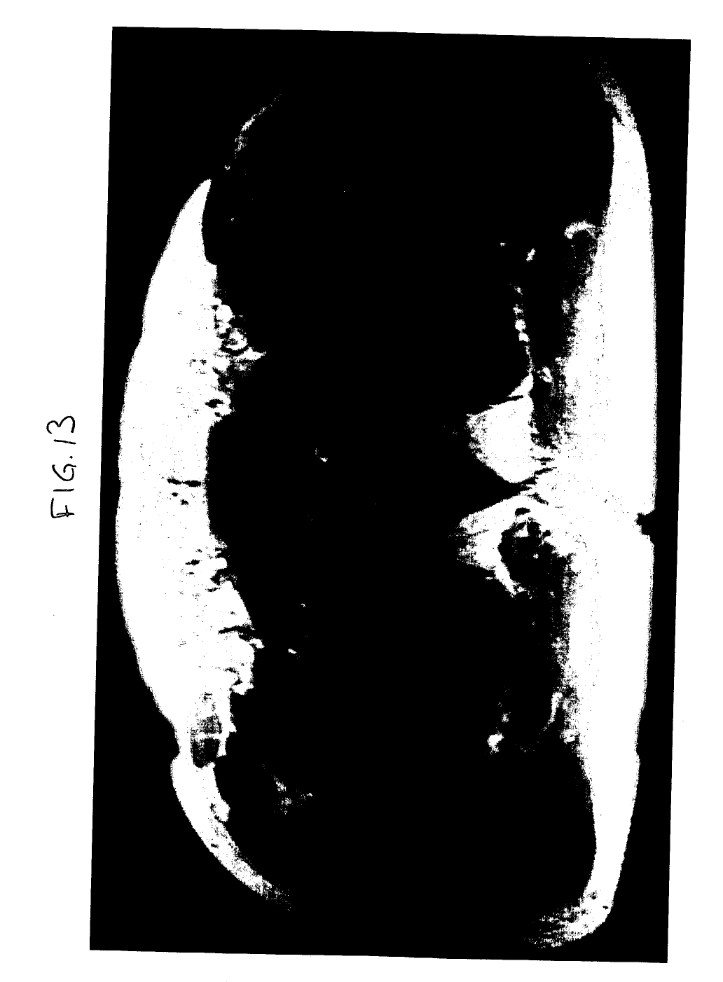

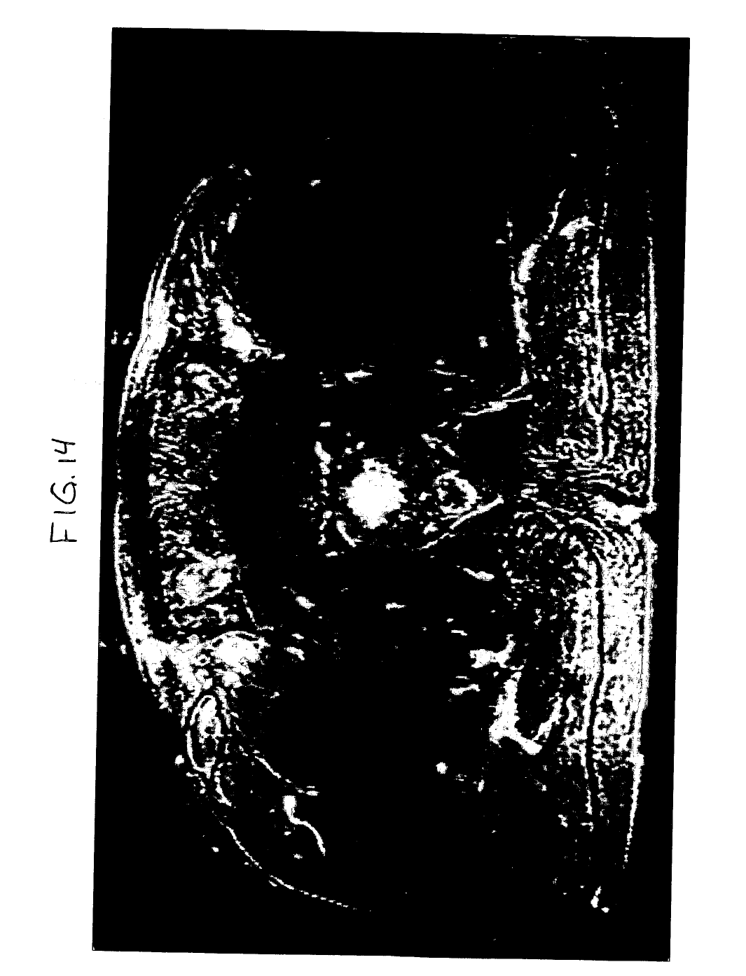

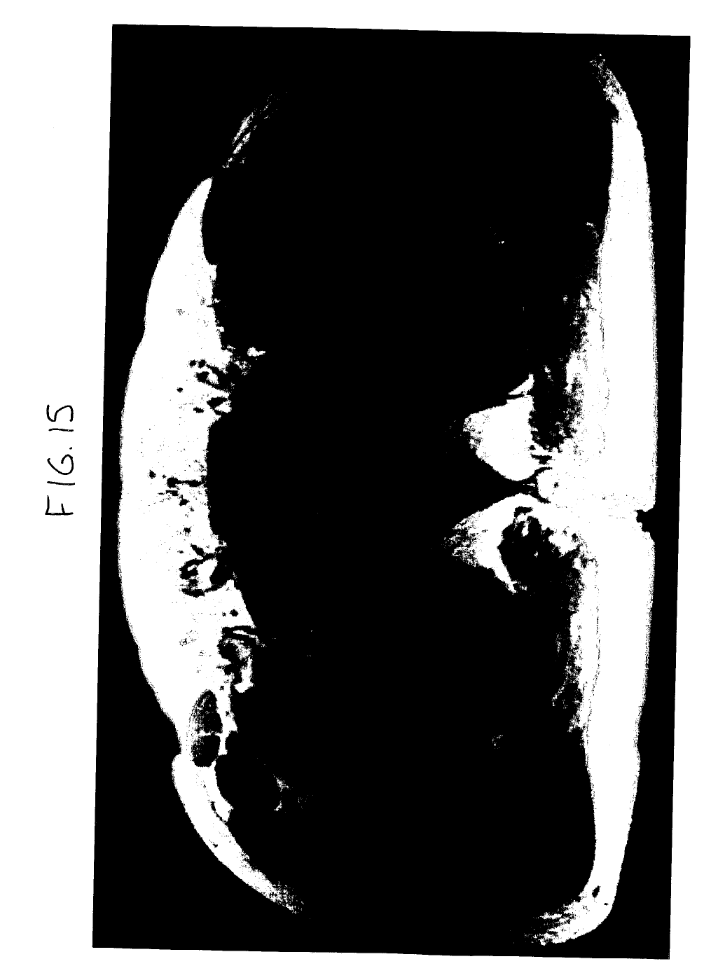

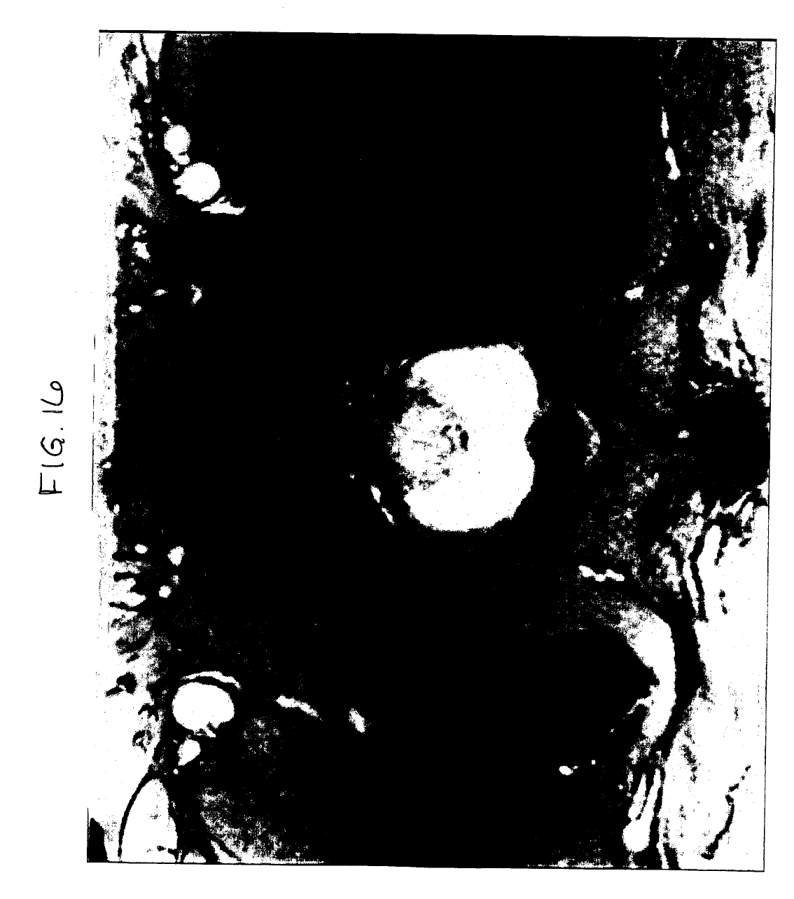

The prostate of an individual was examined using MRI in accordance with the methods of the present invention. FIGS. 5-16 depict sample MRI images of an individual who did not show any region suspicious for cancer according to the methods of the present invention. FIG. 5 is a 1 minute post contrast image. FIG. 6 is a 1 minute subtraction image wherein the image data from the pre contrast image was subtracted from that obtained in the 1 minute post contrast image. FIG. 7 is a 2 minute post contrast image. FIG. 8 is a 2 minute subtraction image wherein the image data from the pre contrast image was subtracted from that obtained in the 2 minute post contrast image. FIG. 9 is a 3 minute post contrast image. FIG. 10 is a 3 minute subtraction image wherein the image data from the pre contrast image was subtracted from that obtained in the 3 minute post contrast image. FIG. 11 is a 4 minute post contrast image. FIG. 12 is a 4 minute subtraction image wherein the image data from the pre contrast image was subtracted from that obtained in the 4 minute post contrast image. FIG. 13 is a 5 minute post contrast image. FIG. 14 is a 5 minute subtraction image wherein the image data from the pre contrast image was subtracted from that obtained in the 5 minute post contrast image. FIG. 15 is a pre contrast image. FIG. 16 is a T2 weighted image.

### Example 6

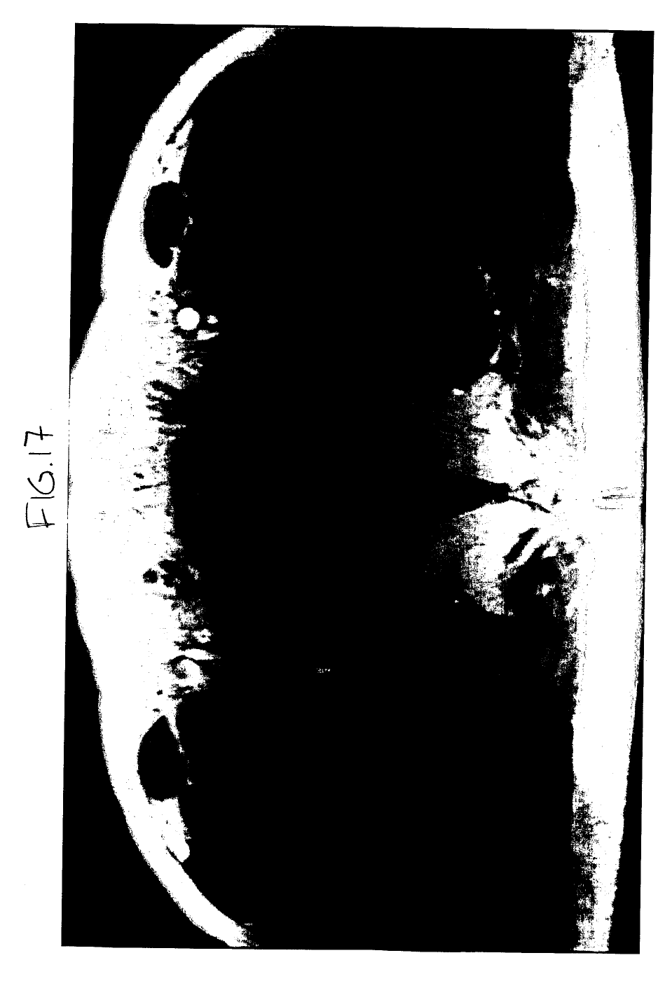

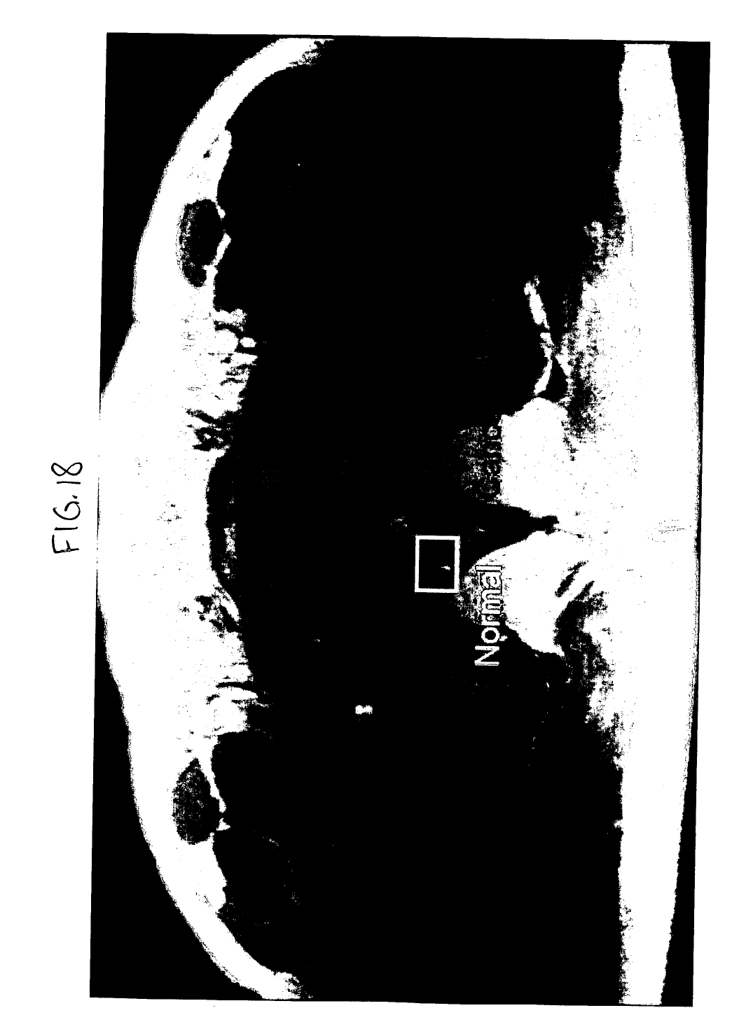

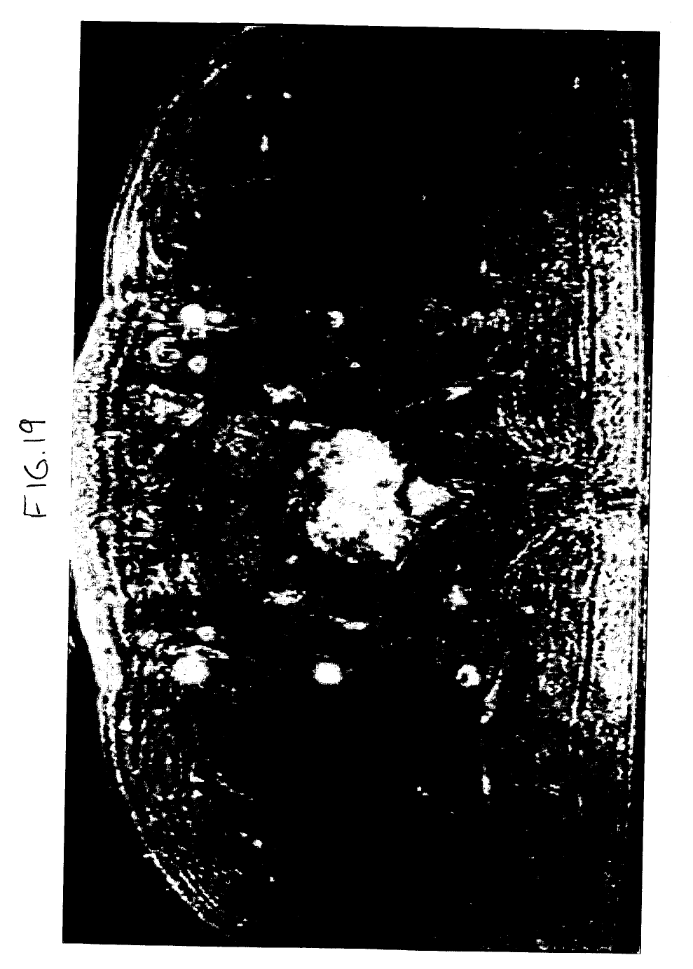

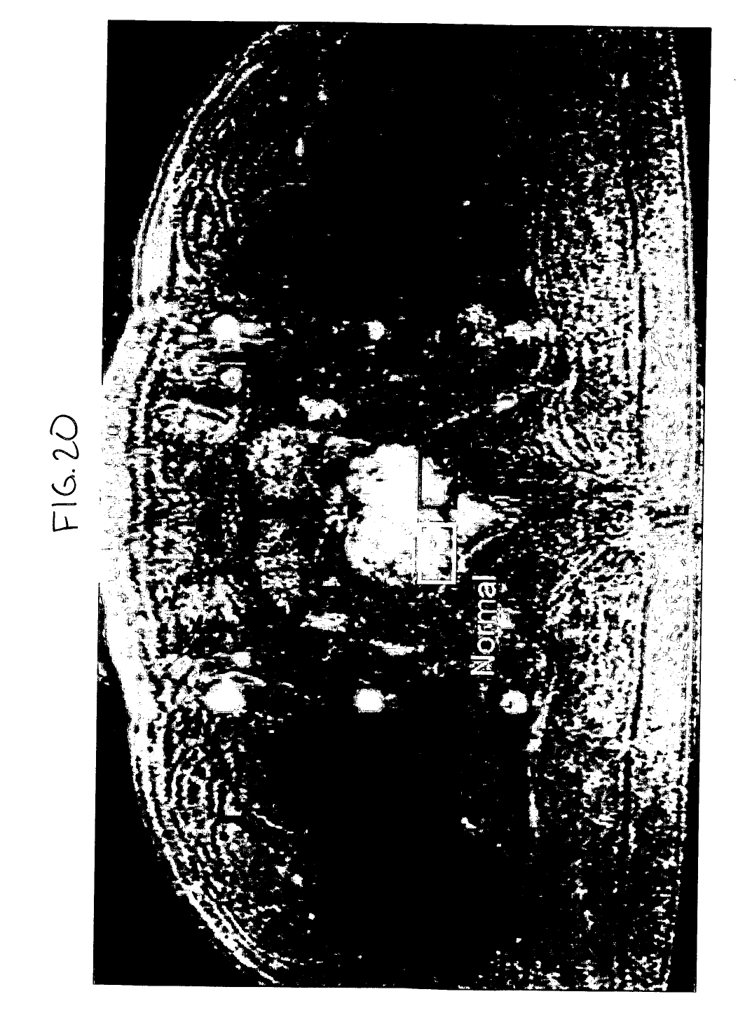

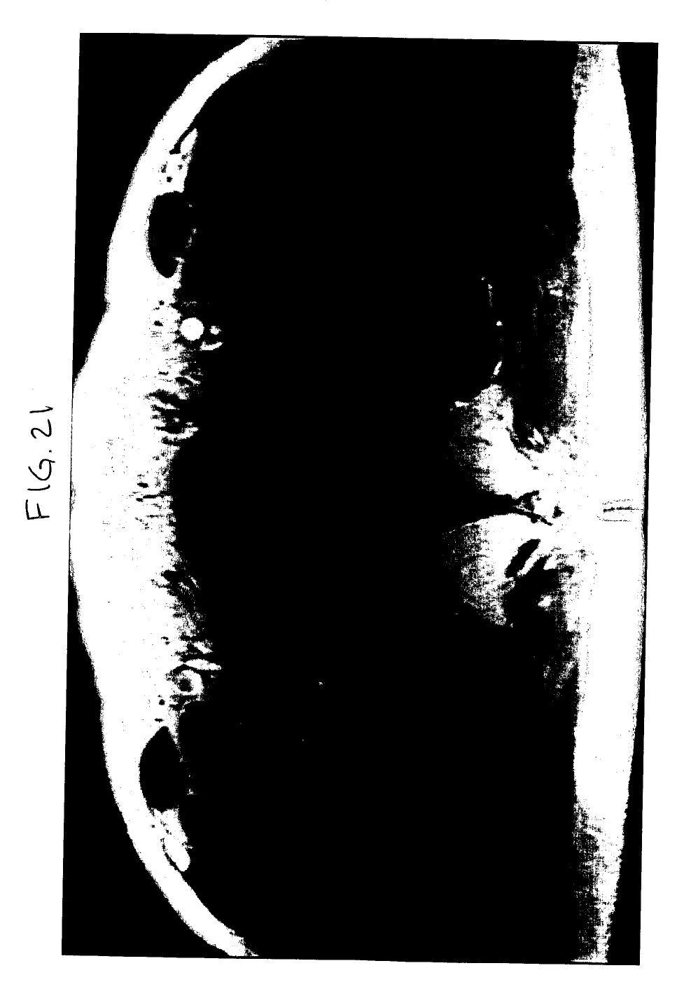

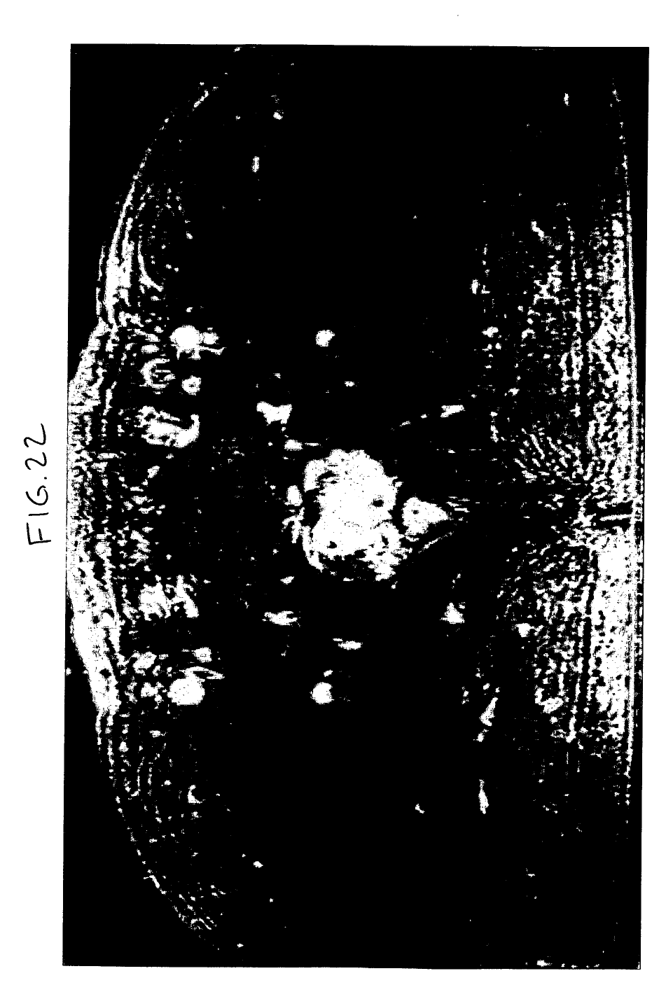

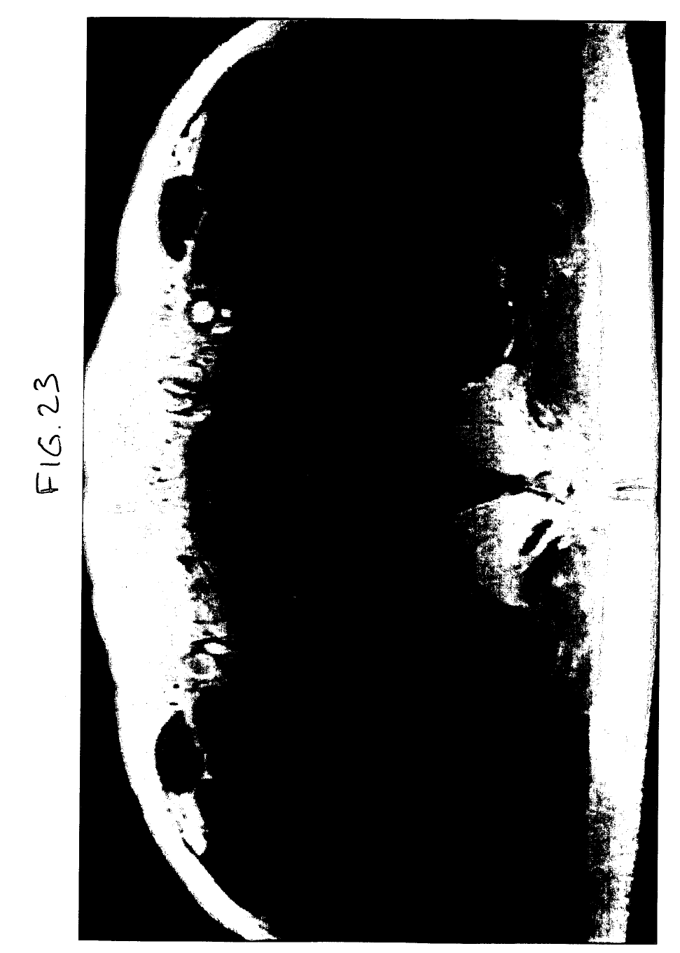

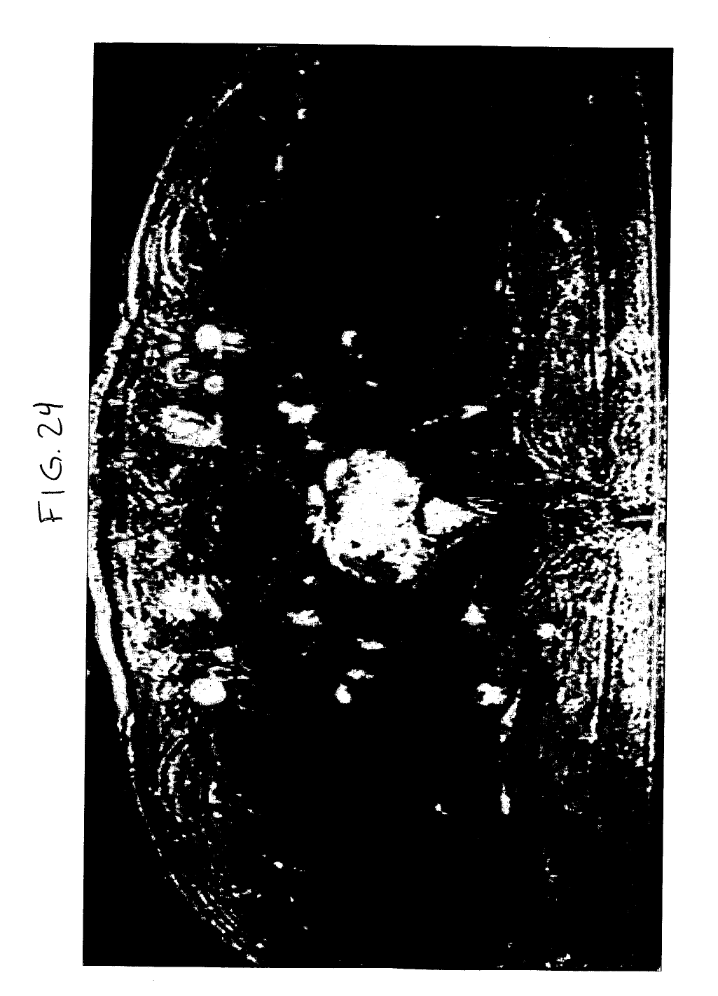

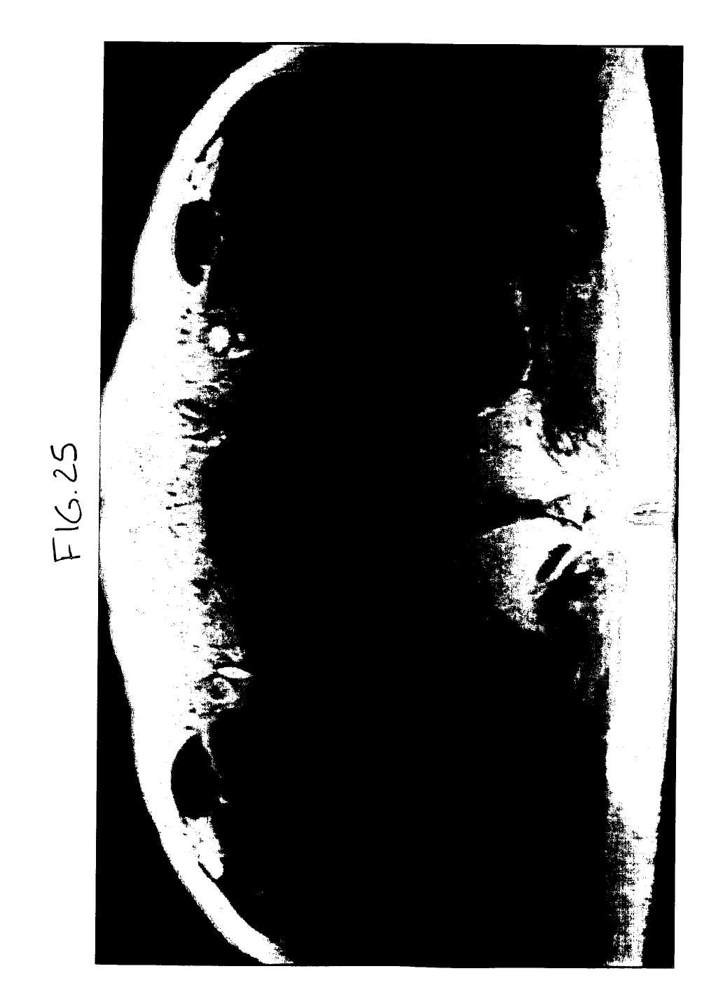

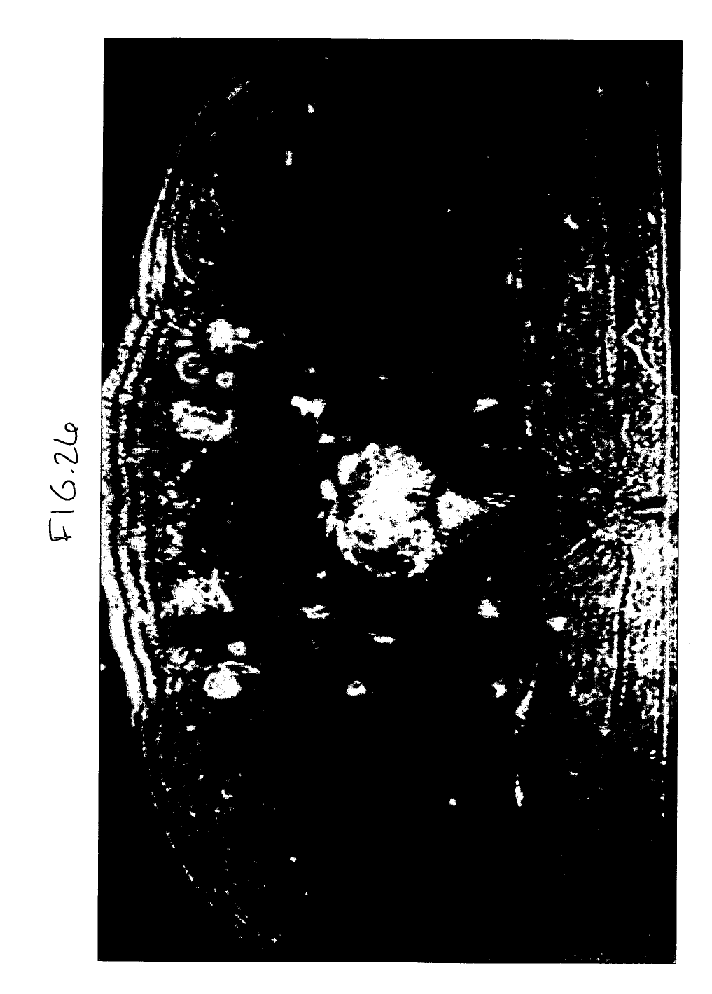

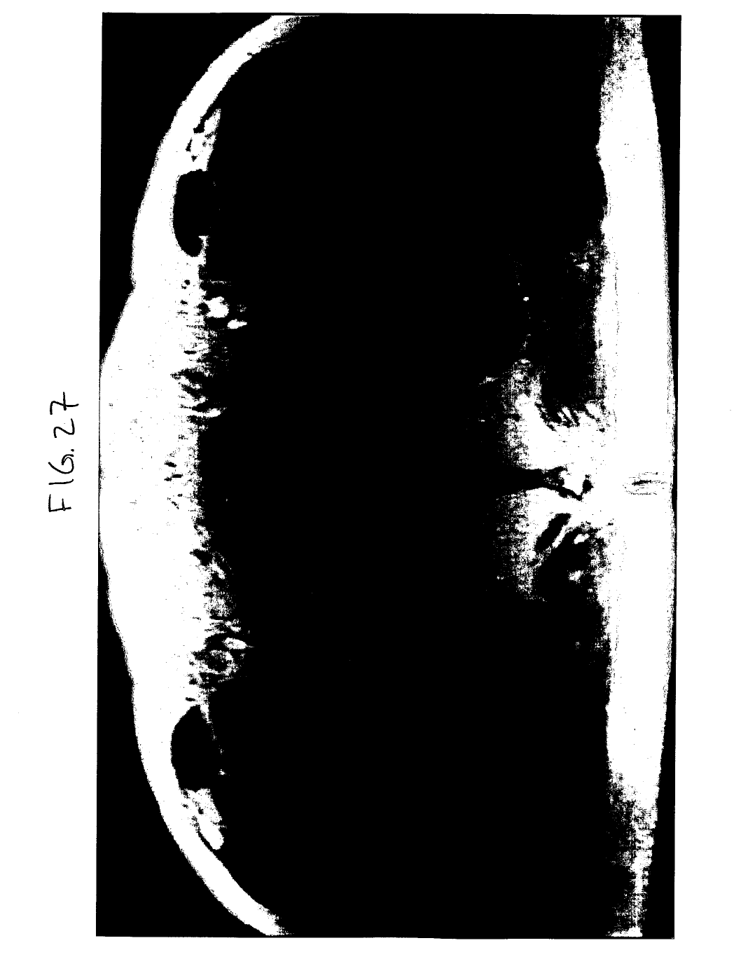

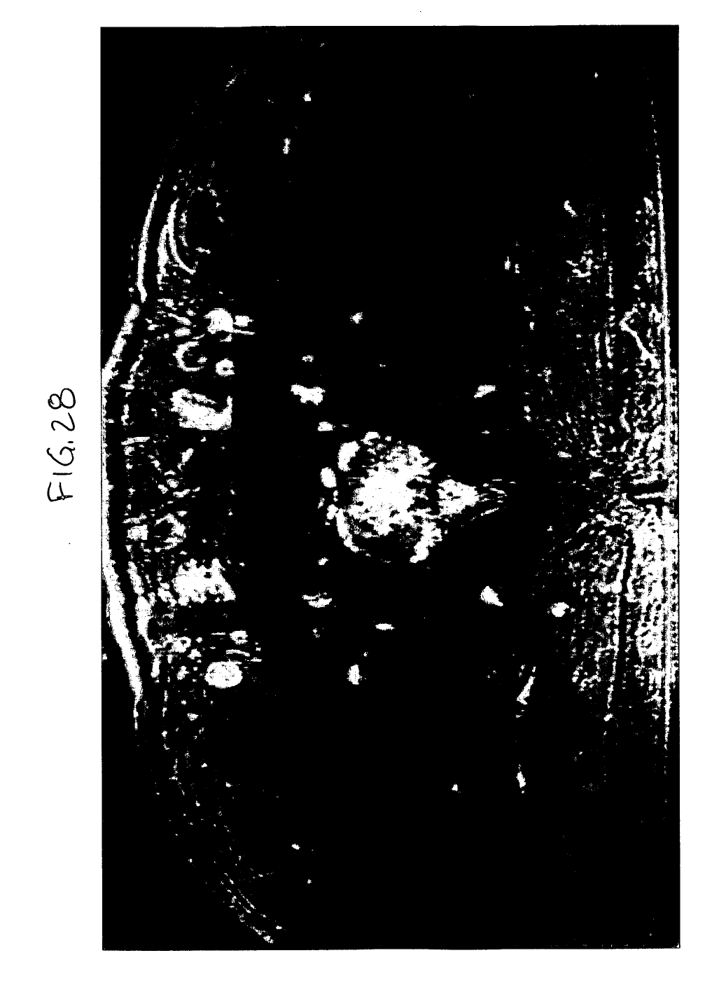

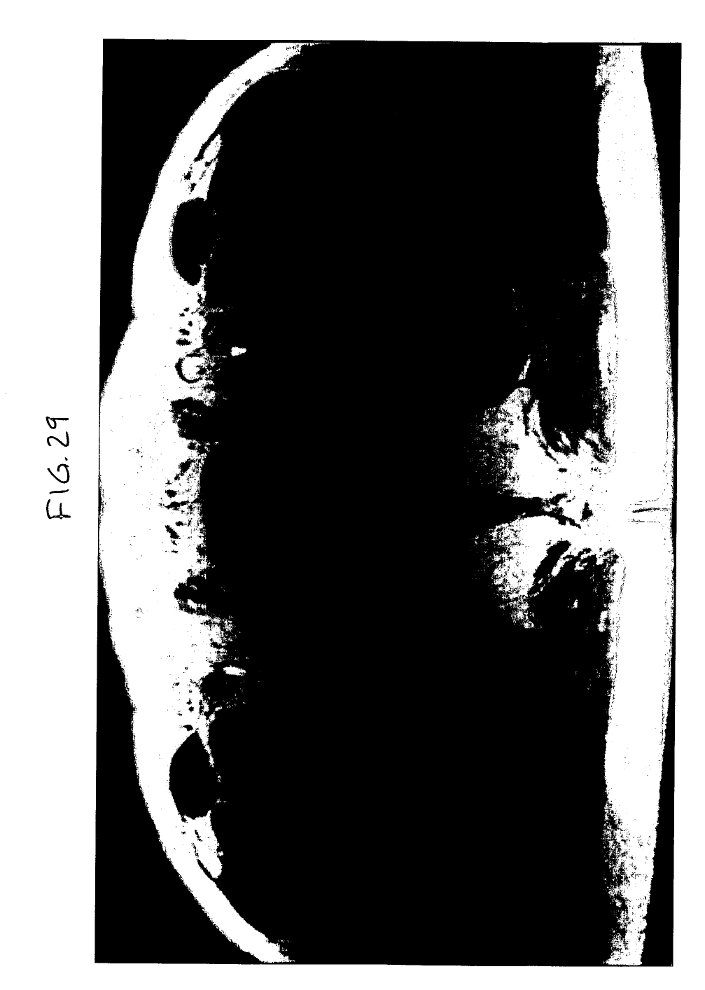

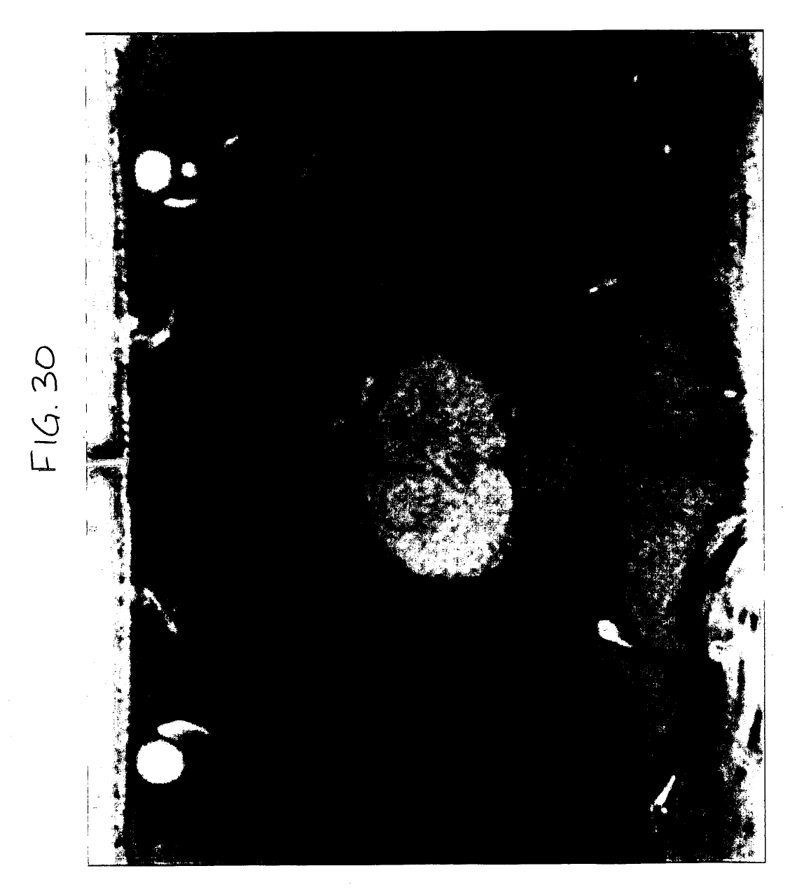

The prostate of an individual was examined using MRI in accordance with the methods of the present invention. FIGS. 17-30 depict sample MRI images of an individual according to the methods of the present invention. This individual had prostate cancer. FIG. 17 is a 1 minute post contrast image. FIG. 18 is a 1 minute post contrast image demonstrating a region of normal prostate tissue and an area with abnormal contrast enhancement indicative of prostate cancer. FIG. 19 is a 1 minute subtraction image wherein the image data from the pre contrast image was subtracted from that obtained in the 1 minute post contrast image. FIG. 20 is a 1 minute subtraction image demonstrating a region of normal prostate tissue and an area with abnormal contrast enhancement indicative of prostate cancer. FIG. 21 is a 2 minute post contrast image. FIG. 22 is a 2 minute subtraction image wherein the image data from the pre contrast image was subtracted from that obtained in the 2 minute post contrast image. FIG. 23 is a 3 minute post contrast image. FIG. 24 is a 3 minute subtraction image wherein the image data from the pre contrast image was subtracted from that obtained in the 3 minute post contrast image. FIG. 25 is a 4 minute post contrast image. FIG. 26 is a 4 minute subtraction image wherein the image data from the pre contrast image was subtracted from that obtained in the 4 minute post contrast image. FIG. 27 is a 5 minute post contrast image. FIG. 28 is a 5 minute subtraction image wherein the image data from the pre contrast image was subtracted from that obtained in the 5 minute post contrast image. FIG. 29 is a pre contrast image. FIG. 30 is a T2 weighted image. A trained radiologist or other person determined from these images that the scanned individual had prostate cancer.

### Example 7

An MRI spectroscopy examination is performed on an individual, in which the relative concentrations of cellular chemicals and metabolites of the prostate tissues are evaluated. MRI spectroscopy can be performed by either single-voxel or a multi-voxel spectroscopy technique at differing spatial resolutions to provide physiologic information about the relative concentrations of the metabolites citrate, creatine, and choline within the prostatic tissue by measuring the specific resonances for citrate, choline and creatine from small volumes of tissue throughout the prostate. The amount of individual resonance present for each metabolite is related to the concentration of these metabolites and changes in these concentrations can be used to identify prostate cancer from normal prostate tissue and from non-cancerous changes of the prostate tissue. Prostate cancer will demonstrate significantly higher choline levels and significantly lower citrate levels as compared to normal prostatic tissues and benign changes in the prostate. The ratio of these metabolites (e.g., choline/citrate) in regions of cancer have minimal overlap with values from normal prostate tissue and BPH values. Also, the physiologic information obtained from MRI spectroscopy may enable assessment of cancer aggressiveness.

While the principles of this invention have been described in connection with specific embodiments, it should be understood clearly that these descriptions are made only by way of example and are not intended to limit the scope of this invention, in any manner. For example, and without limitation, the methodology described herein can be extended to the diagnosis and monitoring of breast, colon, lung, gall bladder, rectal, pancreatic and oral cancers. Other advantages and features of this invention will become apparent to those skilled in the art.

1. A method for screening individuals for prostate cancer, comprising
performing MRI on one or more individuals; and
determining from the MRI whether the one or more individuals has indications of prostate cancer.

2. The method of claim 1, wherein said method further comprises introducing one or more contrast agents into the one or more individuals before performing MRI.

3. The method of claim 2, wherein the one or more contrast agents comprises paramagnetic metal ions selected from the group consisting of manganese, gadolinium and iron.

4. The method of claim 2, wherein said contrast agent is introduced into a individual by injection.

5. The method of claim 2, wherein said contrast agent is introduced into an individual in an amount of approximately 10-30 mL.

6. The method of claim 2, wherein said contrast agent is introduced into an individual in an amount of approximately 15-25 mL.

7. The method of claim 2, wherein said contrast agent is introduced into an individual in an amount of approximately 20 mL.

8. The method of claim 2, further comprising introducing a physiological saline solution into the individual after introducing the contrast agent, in approximately the same volume as the contrast agent.

9. A method of detecting prostate cancer in individuals, comprising screening individuals for prostate cancer, wherein said screening comprises
performing MRI on one or more individuals; and
determining from the MRI whether the one or more individuals have indications of prostate cancer.

10. The method of claim 9, wherein said method further comprises introducing one or more contrast agents into the one or more individuals before performing MRI.

11. A method of screening individuals for prostate cancer comprising
obtaining a baseline pre-contrast MRI of an entire prostate of an individual;
administering one or more contrast agents to the individual in an amount of approximately 0.07-0.13 mmol/kg body weight of the individual;
performing a dynamically enhanced MRI with T1 weighting of the prostate over a period of approximately 2 to 10 minutes at intervals of approximately every 5 to 90 seconds;
plotting a degree of percentage of MRI signal enhancement over time; and
differentiating regions of cancer growth from normal prostate tissue based on the degree of enhancement.

12. The method of claim 11, wherein the contrast agent is administered to the individual in an amount of approximately 0.85-0.115 mmol/kg body weight of the individual.

13. The method of claim 11, wherein the contrast agent is administered to the individual in an amount of approximately 0.1 mmol/kg body weight of the individual.

14. The method of claim 11, wherein the contrast agent is administered at a bolus rate of approximately 1 to 3 cc/sec.

15. The method of claim 11, wherein the contrast agent is administered at a bolus rate of approximately 2 cc/sec.

16. The method of claim 11, wherein the one or more contrast agents comprises paramagnetic metal ions selected from the group consisting of manganese, gadolinium and iron.

17. The method of claim 16, wherein the one or more contrast agents comprises gadolinium.

18. The method of claim 11, further comprising performing T2 weighted imaging of the prostate.

19. The method of claim 11, further comprising performing proton density weighted imaging of the prostate.

20. A method for screening normal risk, asymptomatic individuals for prostate cancer, comprising
performing MRI on one or more normal risk individuals who are asymptomatic for prostate cancer; and
determining from the MRI whether the one or more individuals has indications of prostate cancer.

21. A method of detecting prostate cancer in normal risk, asymptomatic individuals, comprising screening normal risk individuals who are asymptomatic for prostate cancer, wherein said screening comprises
performing MRI on one or more individuals; and
determining from the MRI whether the one or more individuals has indications of prostate cancer.

22. A method of screening a normal risk, asymptomatic individual for prostate cancer comprising
obtaining a baseline pre-contrast MRI of an entire prostate of an individual;
administering one or more contrast agents to the individual in an amount of approximately 0.07-0.13 mmol/kg body weight of the individual;
performing a dynamically enhanced MRI with T1 weighting of the prostate over a period of approximately 2 to 10 minutes at intervals of approximately every 5 to 90 seconds;
plotting a degree of percentage of MRI signal enhancement over time; and
differentiating regions of cancer growth from normal prostate tissue based on the degree of enhancement.

23. The method of claim 1, wherein said method further comprises
performing MRI spectroscopy on one or more individuals; and
determining if the one or more individuals has indications of prostate cancer.

24. A method for screening individuals for prostate cancer comprising
performing MRI spectroscopy on one or more individuals; and
determining if the one or more individuals has indications of prostate cancer.

25. A method of screening an individual for prostate cancer comprising
obtaining a MRI spectroscopy of a prostate of an individual;
determining relative concentrations of metabolites within prostatic tissue of the prostate by measuring specific resonances of said metabolites from tissue throughout the prostate;
determining ratios of the metabolites in the prostate; and
differentiating regions of cancer growth from normal prostate tissue based on the ratios.

26. The method of claim 25, wherein the metabolites comprise one or more metabolites selected from the group consisting of citrate, creatine and choline.

27. A method of screening individuals having an abnormal digital rectal exam for prostate cancer comprising
performing MRI on one or more individuals having an abnormal digital rectal exam; and
determining from the MRI whether the one or more individuals has indications of prostate cancer.

28. A method of guiding a biopsy to a cancer within the prostate of an individual by
performing MRI on the prostate of the individual; and
guiding the biopsy to a desired location determined by viewing images produced by the MRI.
